(19) United States
(12) Patent Application Publication
(10) Pub. No.: US 2021/0244956 A1
(43) Pub. Date: Aug. 12, 2021

(54) Primates Dementia Treatment Apparatus and Driving Method Thereof

(71) Applicant: Whi Young KIM, Busan (KR)

(72) Inventor: Whi Young KIM, Busan (KR)

(21) Appl. No.: 16/802,587

(22) Filed: Feb. 27, 2020

(30) Foreign Application Priority Data
Feb. 7, 2020 (KR) 10-2020-0014982

(51) Int. Cl. A61N 2/00; A61N 2/02

(52) U.S. Cl. CPC A61N 2/006; A61B 5/0476; A61N 2/02

(57) ABSTRACT

A primates dementia treatment apparatus according to an embodiment of the present invention includes: a sensing unit including sensors that sense a cerebral state and a nerve conduction state of a cerebral cortex; a stimulation pulse output unit outputting a stimulation treatment pulse suitable for a cerebral cortex varied by a disease; and a controller controlling the stimulation pulse output unit to generate and output a stimulation treatment pulse customized for each user to be suitable for a form of a cerebral cortex varied by a disease in accordance with a result of real-time checking the cerebral state on the basis of sensing data of the sensing unit.

### BACKGROUND OF THE INVENTION

### Field of the Invention

The present invention relates to a primates dementia treatment apparatus and a driving method thereof and, more particularly, to a primates dementia treatment apparatus that performs cranial nerve stimulation treatment for various variants of the cerebral cortex of patents such as an Alzheimer's patient, and a driving method thereof.

### Description of the Related Art

Magnetic nerve stimulation has been improved in various ways since it was developed by Barker, et al. in the 1980s. Magnetic nerve stimulation introduces an electric field to a human body by using a magnetic field that varies with time, and has the advantage that it can stimulate a deep and wide area particularly in a non-contact type and non-invasive type, so it is recently actively studied even for brain diseases, nervous and muscular rehabilitation treatments, and urinary incontinence. Magnetic treatment using such magnetic treatment equipment is actively accessed and used in the rehabilitation field because it adjusts the excitement of a brain, and the stimulation effect can be achieved by measuring brain reaction at the stimulated positions, so the magnetic treatment is performed usually for motor areas where biological measurement is possible.

However, when a cranial nerve stimulation apparatus is used for a brain disease, dementia, and depression, the apparatus stimulates only a motor cortex regardless of the conditions of the brain diseases, so pathophysiologic characteristics are not considered, and particularly, prudence is required for brain diseases, and reactivity, plasticity, and connectivity of a demential cortex. In particular, this treatment influences initial brain atrophy in Alzheimer's patients and the distance intensity of a magnetic coil, so overactivity of a cortex and a volume of cerebrospinal fluid increased by brain atrophy changes the characteristics of brain tissues and the current induced in the magnetic stimulation device has an adverse influence.

Further, a stimulated portion biochemically and metabolically invades areas other than the motor cortex and a change in the motor cortex area causes problems later. In other words, when magnetic stimulation is used for dementia, only a motor cortex is stimulated regardless of the progression state of the disease, so there is a high possibility that the initial pathophysiologic characteristics are not considered in the progression, and accordingly, there is a problem that it is impossible to know the state of portions except for the motor cortex.

Further, variations of motor areas that use a functional magnetic resonance image before and after stimulation through motor leaning for measuring the result on a nerve system by a stimulation effect in a patient with cerebral infraction may cause plastic variations.

### DOCUMENTS OF RELATED ART

(Patent Document 1) Korean Patent No. 10-0936914 (2010 Jan. 7)

(Patent Document 2) Korean Utility Model No. 20-0428468 (2006 Oct. 2)

(Patent Document 3) Korean Patent No. 10-0924984 (2009 Oct. 28)

(Patent Document 4) Korean Patent No. 10-1733104 (2017 Apr. 27)

### SUMMARY OF THE INVENTION

An objective of the present invention is to provide a primates dementia treatment apparatus that performs cranial nerve stimulation treatment for various variants of the cerebral cortex of patents such as an Alzheimer's patent, and a driving method thereof.

A primates dementia treatment apparatus according to an embodiment of the present invention includes: a sensing unit including sensors that sense a cerebral state and a nerve conduction state of a cerebral cortex; a stimulation pulse output unit outputting a stimulation treatment pulse suitable for a cerebral cortex varied by a disease; and a controller controlling the stimulation pulse output unit to generate and output a stimulation treatment pulse customized for each user to be suitable for a form of a cerebral cortex varied by a disease in accordance with a result of real-time checking the cerebral state on the basis of sensing data of the sensing unit.

The controller may check a form of a cerebral cortex and a disease progression state as the cerebral state and the nerve conduction state on the basis of the sensing data.

The sensing unit may include an electroencephalogram (EEG) sensor that senses the cerebral state and an electromyography (EMG) sensor that senses the nerve conduction state.

The stimulation pulse output unit may include a DC superposition network that superposes and delays the stimulation treatment pulse to fit to a depth and an area of a cerebral cortex of each user in order for treatment according to a reduction of a brain size.

The stimulation pulse output unit may further include a stimulation coil unit that discharges the stimulation treatment pulse of the DC superposition network under the control of the controller.

A method of driving a primates dementia treatment apparatus according to an embodiment of the present invention includes: sensing a cerebral state and a nerve conduction state of a cerebral cortex by means of a sensing unit; outputting a stimulation treatment pulse suitable for a cerebral cortex varied by a disease by means of a stimulation pulse output unit; and controlling the stimulation pulse output unit to generate and output a stimulation treatment pulse customized for each user to be suitable for a form of a cerebral cortex varied by a disease in accordance with a result of real-time checking the cerebral state on the basis of sensing data of the sensing unit by means of a controller.

The controlling may include checking a form of a cerebral cortex and a disease progression state as the cerebral state and the nerve conduction state on the basis of the sensing data.

The sensing may sense the cerebral state using an electroencephalogram (EEG) sensor and senses the nerve conduction state using an electromyography (EMG) sensor.

The outputting of a stimulation treatment pulse may include superposing and delaying the stimulation treatment pulse to fit to a depth and an area of a cerebral cortex of each user in order for treatment according to a reduction of a brain size, using a DC superposition network of the stimulation pulse output unit.

The outputting of a stimulation treatment pulse may discharge the stimulation treatment pulse of the DC superposition network under the control of the controller, using a stimulation coil unit of the stimulation pulse output unit.

According to an embodiment of the present invention, the volume of cerebrospinal fluid increased by brain atrophy can treat cranial nerve stimulation according to a change in characteristic of a brain tissue such that areas except for the motor cortex are not biochemically and metabolically invaded.

Further, according to an embodiment of the present invention, it is possible to achieve an integrated apparatus that operates and connects in real time an EEG sensor and a cranial nerve stimulation apparatus that can stimulate only a target cortex and accurately reflects pathophysiologic characteristics by achieving a customized treatment pulse in accordance with the form of a cerebral cortex and a disease progression state, using a cranial nerve stimulation apparatus for a brain disease and dementia.

Further, according to an embodiment of the present invention, a cranial nerve stimulation apparatus is configured to operate with a cell phone, a computer, a web, etc. by fundamentally introducing monitoring to monitor a disease in real time using peripheral-central Bluetooth for radio communication, and a sensor and a stimulation coil can be added depending on necessity of wire-wireless communication, whereby it is possible to achieve accurate and intense treatment by backing up treatment data.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a diagram showing the entire configuration of a cranial nerve stimulation apparatus according to an embodiment of the present invention;

FIG. 2 is a diagram showing three types that can efficiently use the cranial nerve stimulation apparatus of FIG. 1;

FIG. 3 is a block diagram showing an example of the detailed structure of a cranial nerve stimulation apparatus according to another embodiment of the present invention;

FIG. 4 is an exemplary circuit diagram of the cranial nerve stimulation apparatus of FIG. 3;

FIG. 5 is a diagram showing the hardware configuration of the cranial nerve stimulation apparatus of FIG. 1 according to an embodiment of the present invention;

FIG. 6 is a diagram showing an example when the cranial nerve stimulation apparatus of FIG. 1 according to an embodiment of the present invention is configured in a 2-loop type;

FIG. 7 is a diagram showing an example when the cranial nerve stimulation apparatus of FIG. 1 according to an embodiment of the present invention is configured in a 1-loop type;

FIG. 8 is a waveform diagram showing examples of stimulation pulses of the cranial nerve stimulation apparatus of FIG. 1;

FIG. 9 is a diagram showing Bluetooth communication of FIG. 2;

FIG. 10 is a diagram showing an example of a first operation of the cranial nerve stimulation apparatus of FIG. 1;

FIG. 11 is a diagram showing another example of the configuration of the cranial nerve stimulation apparatus of FIG. 1;

FIG. 12 is a diagram showing another example of the configuration of the cranial nerve stimulation apparatus of FIG. 1;

FIG. 13 is a diagram showing two kinds of signal processing processes for signal analysis;

FIG. 14 is a diagram showing the structure of a web-based monitoring system; and

FIG. 15 is a flowchart showing a driving process of a cranial nerve stimulation apparatus according to an embodiment of the present invention.

### DETAILED DESCRIPTION OF THE INVENTION

Hereinafter, embodiments of the present invention will be described in detail with reference to the accompanying drawings.

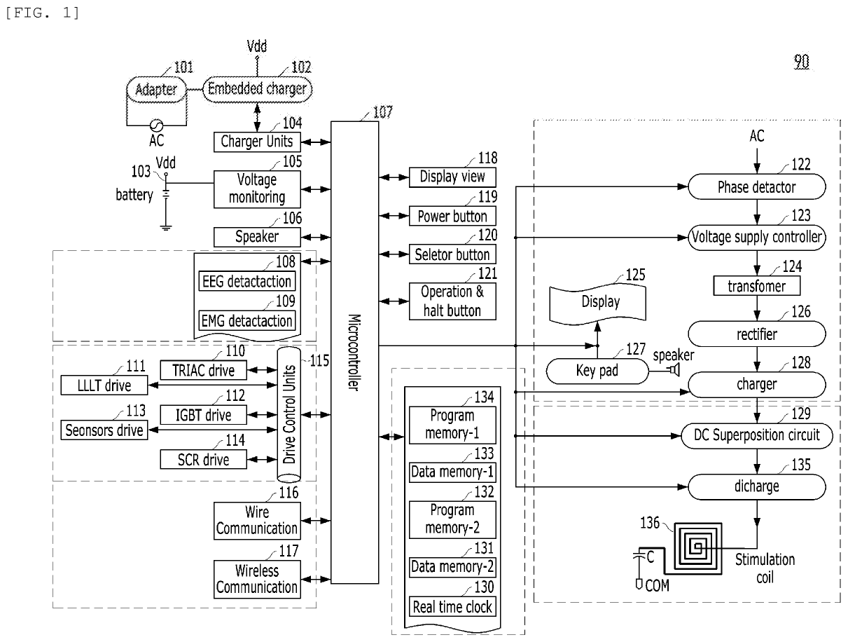

A representative characteristic of Alzheimer's dementia is cerebral cortex atrophy, so a cranial nerve stimulation apparatus using treatment pulses suitable for a varied cerebral cortex is proposed in FIG. 1.

FIG. 1 is a diagram showing the entire configuration of a cranial nerve stimulation apparatus according to an embodiment of the present invention.

As shown in FIG. 1, a cranial nerve stimulation apparatus (or a primates dementia treatment apparatus) 90 according to an embodiment of the present invention includes some or all of a voltage unit 101˜105, a sound output nit 106, a controller 107, a sensor (or sensing) unit 109, a sensor driving unit (or sensor module) 110˜115, a communication unit 116, 117, a user interface 118˜121, an AC voltage generator 122˜128, a memory unit 130˜134, and a stimulation pulse output unit 129, 135, 136.

The term ‘including some or all” means that the cranial nerve stimulation apparatus 90 is configured without some components such as the sound output unit 106 or some components such as the memory 130˜134 are included in other components such as the controller 107, and it is exemplified in the following description that all of them are included to help sufficiently understand the present invention.

The voltage unit 101˜105 can use voltage required for the cranial nerve stimulation apparatus or DC voltage of a battery by receiving and converting AC voltage into DC voltage. The voltage unit 101˜105 includes an adapter 101 for using common power, a charger 102 that performs even a built-in type charging function when it is used as a portable device, a power battery 103 for portable use, a charging port 104 that is controlled by the controller 107 such as a microprocessor, and a voltage monitoring unit 105 that performs a voltage monitoring function that is controlled by the microprocessor. The voltage monitoring unit 105 can measure a remaining voltage of the battery 103.

The sound output unit 106 includes a speaker and outputs a sound to the outside.

The controller 107 includes a microprocessor such as a CPU or an MPU, as shown in FIG. 1. The microprocessor is a large-scale integration (LSI) that performs important operations such as logic, determination, and calculation in cooperation with various peripheral circuits. A microprocessor is usually composed of an IC memory in which software is stored around a CPU and an I/O interface for transmitting/receiving data to/from the CPU and peripheral circuits. The controller 107 generally controls the operations of various components including the sensor unit 108, 109 of the cranial nerve stimulation apparatus 90. Representatively, the controller 107 can accurately reflect pathophysiologic characteristics by generating customized treatment pulses in accordance with the form of a cerebral cortex and the progress state of a disease and can estimate continuity with cerebral physiological variations in areas except for the motor cortex, and for this purpose, the controller can be operated in real time in cooperation with an EEG sensor of the sensor unit 108, 109.

The sensor unit 108, 109 includes a first sensor unit 108 and a second sensor unit 109. The first sensor unit 108 may include an Electroencephalogram (EEG) sensor as a brain-muscle power input sensor as an option and the second sensor unit 109 includes an ElectroMyoGraphy (EMG) sensor, receives data from the microprocessor through the EEG sensor and the EMG sensor, and performs the followings. The EEG sensor is a brain wave sensor and determines a cranial nerve signal. On the other hand, the EMG sensor, which is an electromyograph sensor, measures action potential of muscles. Electromyography is a method of measuring a state change of a muscle by examining electrical activity of the muscle that is controlled by nerves and in which a fine current always flows.

The sensor driving unit (or a brain state measurer or a sensor driving module) 110˜115 includes some or all of a triac driving unit 110, a low-power laser treatment unit (LLLT) 111, an Insulated Gate Bipolar Transistor (IGBT) 112, various sensor driving units 113, an SCR driver 114, and a driving control unit 115. The driving control unit 115 can individually control the triac driving unit 110, the low-power laser treatment unit 111, the IGBT 112, the various sensor driving units 113, and the SCR driver 114.

The communication unit 116, 117 may include a first communication unit 116 and a second communication unit 117. The first communication unit 116 is a wire communication port and the second communication unit 117 can perform a wireless communication port function.

The user interface 118˜121 includes a display view (or display) 118, a power button 119, a sensor button 120, and a start/stop button 120. A user can input power, start an operation, and stop an operation by selecting various buttons and the display view 118 is a display screen and can display various images or the operation state of a device.

The AC voltage generator 122˜128 may include some or all of a phase detector 122 that detects a phase coming with AC, a voltage adjustment controller 123, a transformer 124 for increasing voltage, a display window 125 that can be seen outside the primates dementia treatment apparatus 90, a rectifier 126 that rectifies voltage coming out of the transformer 124, a keypad 127 allowing for input through the display window (or touch screen) 125, and a charger 128 for storing a rectified voltage. The voltage adjustment controller 123 can be controlled to perform PWM control, etc. by the microprocessor.

The memory unit 130˜134 may include some or all of a real-time clock (generation) unit 130, a data memory 131, a program memory 132, an assistant data memory 133, and an assistant program memory 134. A real-time clock may be used to show start of data processing, etc. The memory unit 130˜134 stores data and can store various predetermined data related to stimulation pulse generation in accordance with an embodiment of the present invention.

The stimulation pulse output unit 129, 135, 136 includes some or all of a superposition network 129 that superposes or delays a discharge pulse to be suitable for treatment according to contraction of brain size, which is a characteristic of Alzheimer's disease, by using a DC superposition network, a discharge circuit 135, and a stimulator 136 that functions as a final load using a principle of performing treatment by applying a corresponding pulse according to the depth and area of a cerebral cortex using a coil operating a superposed stimulation pulse for treatment.

In an embodiment of the present invention, for treatment suitable for the form of a varied cerebral cortex such as the size and the shape of the brain of a brain disease target such as an animal or a human, it may be considered that the cranial nerve stimulation apparatus 90 that checks in real time a cerebral state using an EEG sensor for checking a cerebral state and an EMG sensor being able to check a nerve conduction state, performs real-time monitoring using optimal treatment pulses and an optimal pulse forming apparatus, which are suitable for the form of a cerebral cortex varied by a disease, and EEG, EMG, a cell phone, a computer, a web, etc., and generates suitable treatment pulses for a cerebral cortex varied by a brain disease for various purposes.

As a result, an embodiment of the present invention may recover the functions of a brain using a neural net that is not damaged when a portion of a brain is damaged through magnetic stimulation, which may be a treatment method that can induce functional recovery in clinical tests designed to recover a brain. In particular, it is required to delay, diagnose, and treat deterioration of an irreversible recognition function and changes in normal life function and neuropsychiatric behaviors that gradually progress in, a brain disease, dementia, and depression, and it is required to diagnose the conditions before a critical symptom appears. Further, according to many studies, it has been proved that when a partial change of a cerebral cortex by a magnetic stimulation is applied to a primary motor area, excitement of the corticospinal tract changes for several minutes to several hours.

That is, the cranial nerve stimulation apparatus 90 according to an embodiment of the present invention may be generally configured such that a treatment stimulation coil of the cranial nerve stimulation apparatus 90 is attachable, thus it is possible to control EEG and EMG sensors equipped with LEDS showing a problem in a lesion and a pulse forming apparatus for generating treatment pulses suitable for a varied cerebral cortex of a patient with a brain disease, and a magnetic coils can be replaced or added, depending on the performance and capacity of a power device.

EEG and EMG sensor modules are composed of an energy harvester and EEG and EGM front ends (analog) and are formed to be light and small for the convenience of carrying and moving. The microcontroller includes an A/D converter, a memory, and a Universal Asynchronous Receiver/Transmitter (UART) module, thereby having an advantage that power consumption can be reduced. Further, a microprocessor module (which is, for example, replaceable depending on DSP&FPGA performance) may be additionally used.

However, when the cranial nerve stimulation apparatus 90 is used for a brain disease and dementia, the device stimulates only a motor cortex regardless of the conditions of the brain diseases, so pathophysiologic characteristics are not considered, and prudence is required for brain diseases, and reactivity, plasticity, and connectivity of a demential cortex. Further, since brain atrophy itself is in connection with overactivity of a cortex, the volume of cerebrospinal fluid changes the characteristics of brain tissues and influences an induced current, and as problems with a stimulated portion, according to biochemical and metabolic studies for Alzheimer's dementia, areas except for a motor cortex is invaded in an early stage and the areas of the motor cortex is shown later. Accordingly, the levels of amyloid-β and t-Tau protein in cerebrospinal fluid influence the effect of cranial nerve stimulation, and according to Koch, etc., when the level of t-Tau is high, it is required to increase the stimulation effect applied to an Alzheimer's dementia patient.

Accordingly, it is required to control the levels of amyloid-β and t-Tau protein in cerebrospinal fluid when applying the cranial nerve stimulation apparatus 90, and there is an advantage that it is possible to discriminate in-cortex strengthening and suppression effects by operating the EEG sensor in association with connectivity of a cortex.

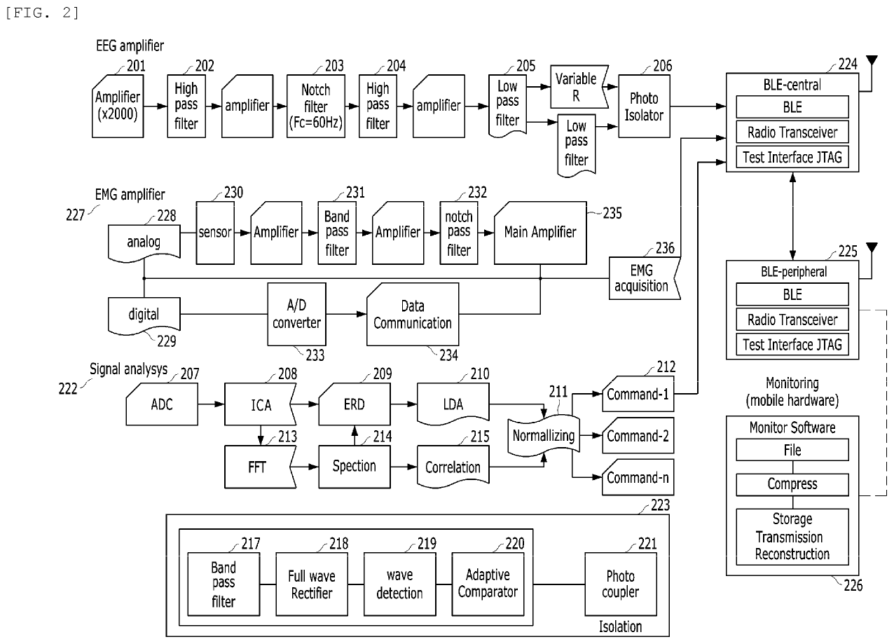

FIG. 2 is a diagram showing three types that can efficiently use the cranial nerve stimulation apparatus of FIG. 1.

In an embodiment of the present invention, as shown in FIG. 1, three types may be exemplified so that the cranial nerve stimulation apparatus 90 using treatment pulses suitable for a varied cerebral cortex can be efficiently used. The first one may be an EEG device that measures brain waves, the second one may be an EMG device 227 that measures the states of muscles, and the third one may be an algorithm 222 that measures the signals and an analysis program 223 having a hardware function, whereby detection waveforms can be configured.

The EEG device may include an amplifier 201 that amplifies waveforms obtained from a brain electrode, a high-band filter 202 that blocks high bands, a notch filter 203 that attenuates specific frequency bands, a high-band filter 204, a low-band filter 205 that filters out low bands, and a photo isolator 206 that transmits signal waveforms obtained at the previous stage to the Bluetooth of a first communication unit 224 without a loss in an insulation state.

The EMG device 227 includes a process of an EMG analog signal 228, a digital receiver 229, a metal sensor 230, a band-pass filter 231, a notch filter 232 that attenuates specific frequency bands, a converter 233 that converts an analog signal into a digital signal, a data communication (unit) 234, an amplifier 235 that amplifies a fine signal 235, thereby finally obtaining an EMG signal (236).

The signal processors 222 and 223 include the concepts of a signal analysis algorithm 222 and a signal analysis process hardware 223. A converter 297 that converts an analog signal into a digital signal, an Independent Component Analysis (ICA) (unit) 208, an ERD 209, an LDA 210, a normalizing (unit) 211, a command (unit) 212, a Fast Fourier Transform (FFT) (unit) 213, a spection (unit) 214, a correlation unit 215, a band-pass filter 217, an electric wave rectification (unit) 218, a waveform detection (unit) 219, a comparison adjustment (unit) 220, and a photo coupler 221 may be included.

Further, the cranial nerve stimulation apparatus 90 includes a first communication unit 224, a second communication unit 225, and a monitoring unit 226, the first communication unit 224 and the second communication unit 225 includes Bluetooth communication modules for transmission/reception, and the monitoring unit 226 has a process of monitoring to a mobile hardware, which may be monitored by a cell phone such as a smartphone.

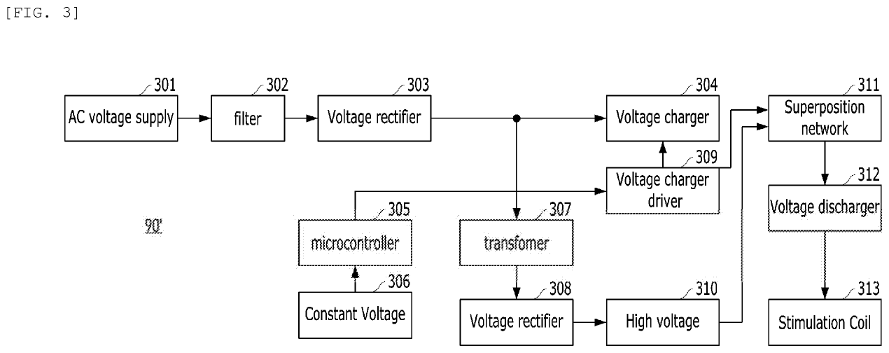

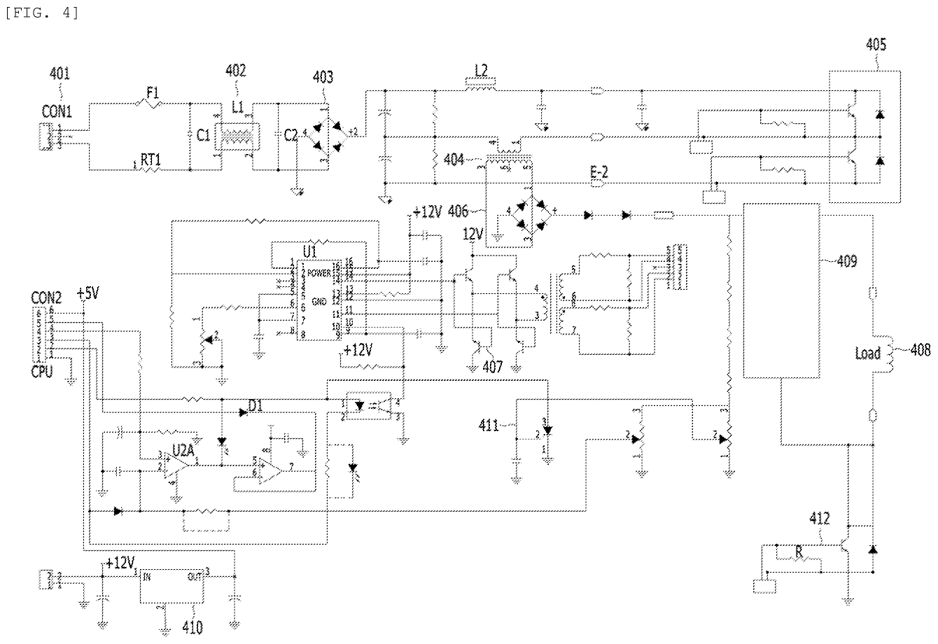

FIG. 3 is a block diagram showing an example of the detailed structure of a cranial nerve stimulation apparatus according to another embodiment of the present invention and FIG. 4 is an exemplary circuit diagram of the cranial nerve stimulation apparatus of FIG. 3.

As shown in FIGS. 3 and 4, the cranial nerve stimulation apparatus 90′ according to another embodiment of the present invention shown in FIG. 1 includes some or all of a voltage supplier 301, a filter 302, a first rectifier 303, chargers 304 and 309, a controller 305, a constant voltage unit 306, a transformer 307, a second rectifier 308, a high-voltage generator 310, a superposition network 311, a discharger 312, and a stimulator 313, in which the term “includes some or all” means the same as the above description.

The voltage supplier 301 supplies AC power and the filter 302 filters out noise, etc. The first rectifier 303 rectifies AC voltage. A bridge circuit may be used, as shown in FIG. 4, for full-wave rectification and half-wave rectification.

The chargers 304 and 309 perform voltage charging and the intensity of the voltage can be adjusted by the controller 305. The voltage charging driver 309 can operate individually with the charger 304 and the superposition network 311. The voltage charging driver 309 may provide a bias voltage for operating the superposition network 311 by controlling the charger 304 in accordance with control of the controller 305.

The controller 305 controls general operations of the components of the cranial nerve stimulation apparatus 90′ and the constant voltage unit 306 may maintain constant voltage. It may be a kind of memory. Alternatively, it may be a reference voltage unit that provides a reference voltage. The transformer 307 amplifies a voltage in proportion to a winding ratio and the second rectifier 308 rectifies the voltage amplified by the transformer 307.

The high-voltage generator 310 makes the rectified voltage of the second rectifier 308 high voltage. Charging pumping, DC-DC converting, or a voltage doubler may be used to generate high voltage. The superposition network 311 achieves an efficient method of performing pulse superposition on a superposition network that is one of important functions in an embodiment of the present invention, the discharger 312 is in charge of discharging a stored voltage through a stimulation coil, and the stimulator 313 performs treatment by discharging a voltage to a stimulation coil that is a final load.

FIG. 4 is an actual analog design drawing of FIG. 3, briefly, generates DC by rectifying a current through an AC input consent 401, an AC filter 402, and diode bridge 403, and drives and operates a transformer 404 for generating a double voltage, an IGBT module 405 being in charge of voltage capacity, a function 406 for generating a high voltage through voltage rectification, and an IGBT 407. It may be possible to generate a high voltage using a secondary voltage of the transformer 404 by turning on/off a switching element of the IGBT 407 configured in a full-bridge type. The treatment stimulation coil 408 is a final load and the superposition network 409 superposes, delays, etc. a discharge pulse to be suitable for treatment by applying a DC superposition network for treatment for a reduction of a brain size that is a characteristic of Alzheimer's disease. The constant voltage circuit 410 generates a constant voltage to be used for a digital part and a state output unit 411 such as a light emission diode shows the voltage that is discharged from the discharger 412. The discharge 410 functions as a load, and accordingly, a stimulation pulse can be output. A pair of conduction wires for outputting a stimulation pulse and an electrode connected to the conduction wires may be included in the discharger 410.

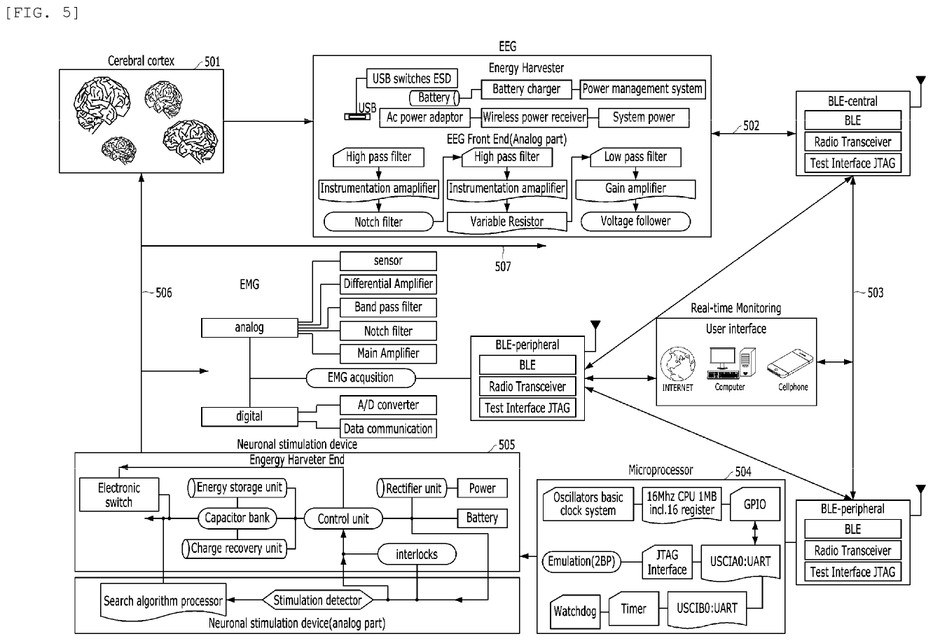

FIG. 5 is a diagram showing the hardware configuration of the cranial nerve stimulation apparatus of FIG. 1 according to an embodiment of the present invention.

FIG. 5 is a fundamental conceptual diagram configured as hardware in accordance with an embodiment of the present invention and includes some or all of a sensor 501, a first communication unit 502, a second communication unit 503, a controller 504, a nerve stimulation device 505, and a stimulation adjuster 506, in which the term “includes some or all” is the same as the meaning described above.

The sensor 501 performs an operation for obtaining a biological information of a treatment pulse that is suitable for a varied cerebral cortex in accordance with an embodiment of the present invention. The first communication unit 502 performs Bluetooth communication and shares and transmits data obtained from a sensor device including the sensor 501, and the second communication unit 503 performs a Bluetooth function for receiving biological information transmitted from the Bluetooth of a transmission function as a peripheral device. The controller 504 includes a microprocessor being in charge of control management and the nerve stimulation device 505 is in charge of stimulation suitable for a cerebral disease. The stimulation adjuster 506 provides more stable stimulation by correcting and selecting stimulation intensity and a superposition pulse through reaction muscles.

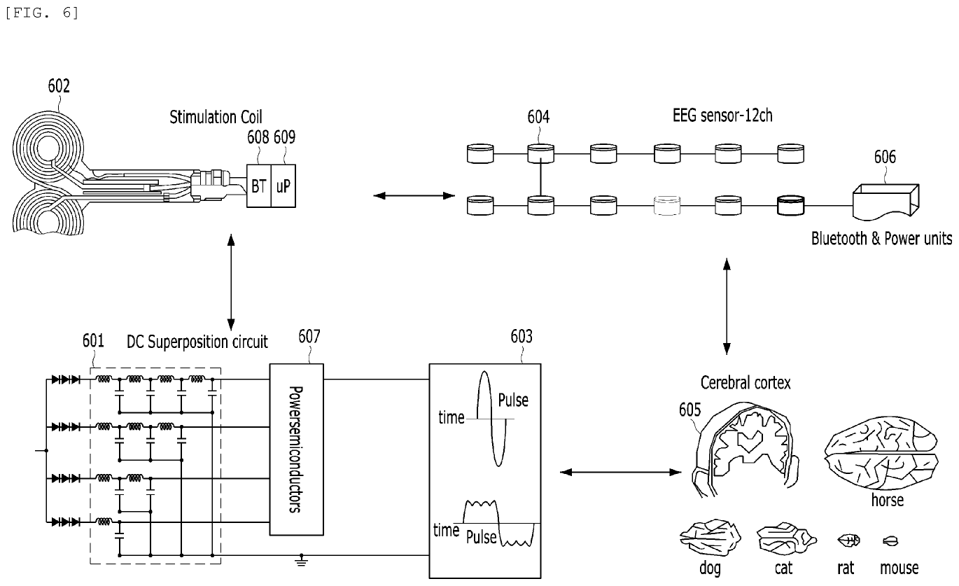

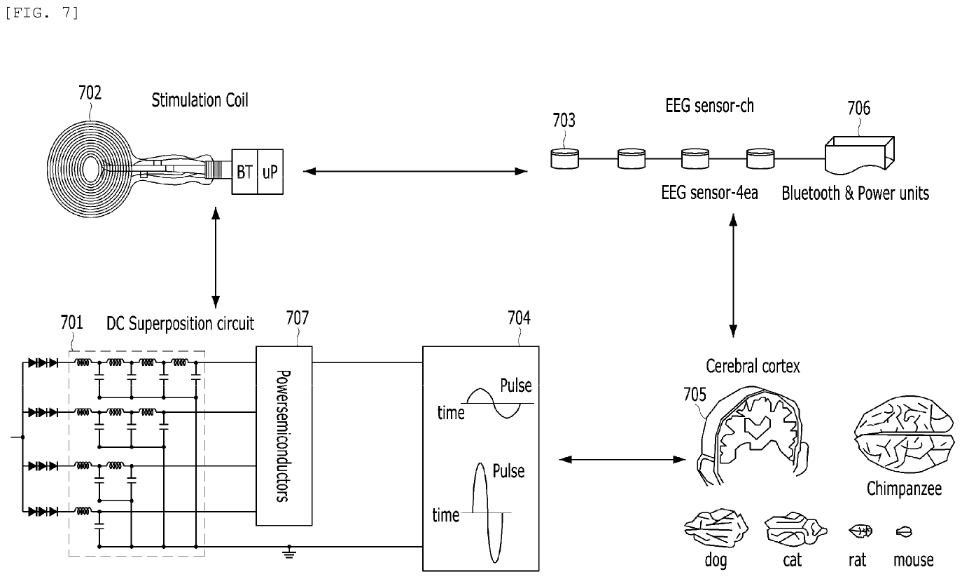

FIG. 6 is a diagram showing an example when the cranial nerve stimulation apparatus of FIG. 1 according to an embodiment of the present invention is configured in a 2-loop type and FIG. 7 is a diagram showing an example when the cranial nerve stimulation apparatus of FIG. 1 according to an embodiment of the present invention is configured in a 1-loop type.

FIG. 6 shows a 2-loop type treatment apparatus and shows a DC superposition circuit 601, a stimulation coil 602 connected with Bluetooth and a microprocessor, and a sample for achieving a necessary stimulation pulse for a disease through DC superposition. As can be seen by reference numeral ‘604’, precision depends on the channel of a brain wave obtained from a cerebral cortex and reference numeral ‘605’ shows a cerebral cortex of mammalia. Reference numeral ‘606’ shows a brain wave measurer connected with Bluetooth and a power device, reference numeral ‘607’ shows a power semiconductor for achieving superposition, reference numeral ‘608’ shows Bluetooth, and reference numeral ‘609’ shows a microprocessor that controls an overall process.

FIG. 7 shows a 1-loop type apparatus, in which reference numeral ‘701’ shows superposition, reference numeral ‘702’ shows a stimulation coil connected with Bluetooth and a microprocessor, reference numeral ‘703’ shows precision that depends on the channel of a brain wave obtained from a cerebral cortex and shows a sample for achieving a necessary stimulation pulse for a disease through superposition.

Reference numeral ‘704’ shows a sample for achieving a necessary stimulation pulse for a disease through superposition. Reference numeral ‘705’ shows a cerebral cortex of mammalia, reference numeral ‘706’ shows a brain wave measurer connected with Bluetooth and a power device, and reference numeral ‘707’ shows a power semiconductor for achieving superposition.

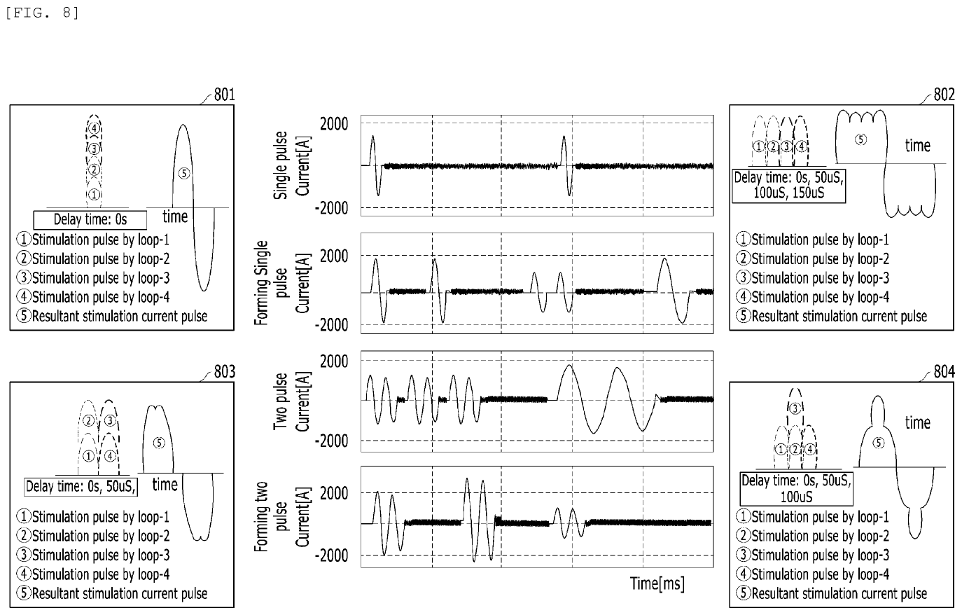

FIG. 8 is a waveform diagram showing examples of stimulation pulses of the cranial nerve stimulation apparatus of FIG. 1.

For the convenience of description, referring to FIG. 8 with FIGS. 6 and 7, in FIG. 8, reference numeral ‘801’ shows a current pulse of a stimulation coil in which s1, s2, s3, and s4 were simultaneously triggered, and the delay time was 0 sec and the pulse width was 240 μs, and reference numeral ‘802’ shows a flat 4-stage current pulse in which s1 was triggered at 0 sec without delay time, s2 was triggered after delay time 50 μs, s3 was triggered after delay time 100 μs, and s4 was triggered after delay time 150 μs, and the stimulation pulse width was 960 μs. Reference numeral ‘803’ shows a 3-stage current pulse of a stimulation coil in which s1 and s2 were simultaneously triggered, and then s3 was triggered at 30 μs and s4 was triggered at 100 μs, and the pulse width was 720 μs. Reference numeral ‘804’ shows a current pulse having a 3-stage shape with a rising center in which s1 was triggered at 0 sec without delay time, and then s2 and s3 are simultaneously triggered after delay time 50 μs and s4 was triggered after delay time 100 μs, and the stimulation pulse width was 720 μs. As a result, it possible to freely change a stimulation pulse with various intensities of output, pulse widths, and pulse shapes and to overcome the limitation of a stimulation treatment pulse by an existing simple sine wave pulse shape.

For this operation, the microprocessor 609 shown in FIGS. 6 and 7 can generate the pulses shown in FIG. 8 by controlling switching elements configured in a bridge type in the DC superposition circuits 601 and 710, in detail, the switching elements of the high-voltage generator 310 shown in FIG. 3 or s1 to s4 of the IGBT (unit) 407 configured in a full-bridge type in FIG. 4 in accordance with a predetermined method, and the detailed control operation may form stimulation pulses in various shapes, depending on what type a control algorithm is configured in. Triggering the switching elements means controlling. The shape of the stimulation pulse may be formed to be suitable for the form of a varied cerebral cortex.

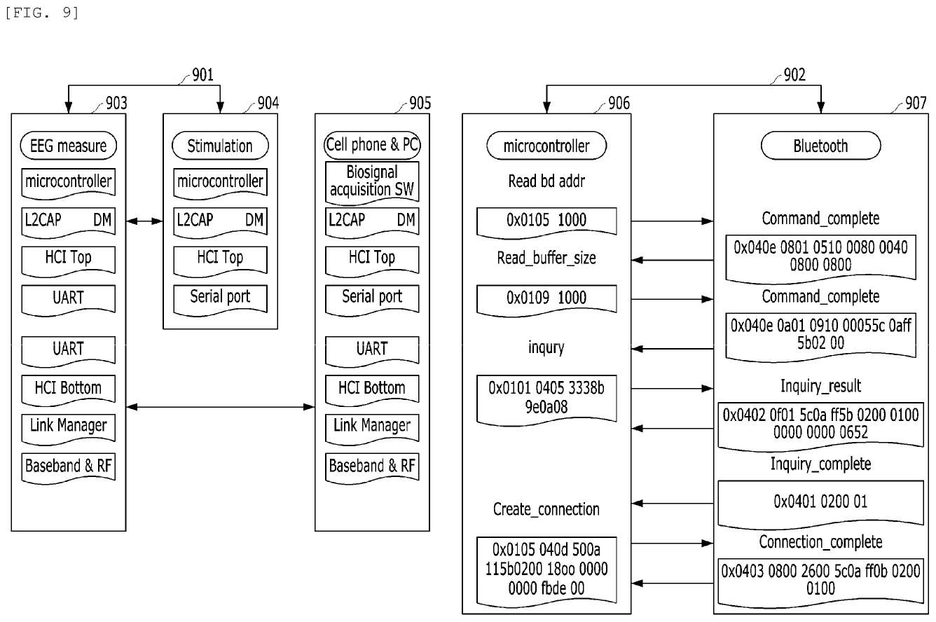

FIG. 9 is a diagram showing Bluetooth communication of FIG. 2.

The communication module of FIG. 2 according to an embodiment of the present invention performs Bluetooth communication, in which, as shown in FIG. 9, an HCI and an L2CAP perform the most important role in Bluetooth protocols and the L2CAP protocol is a protocol that should be fundamentally implemented in Bluetooth. Further, the L2CAP is positioned right over an HCI layer and makes it possible to transmit/receive a data packet up to 64 Kbyte to/from an upper protocol or application. Reference number ‘901’ shows a Bluetooth module equipped with BlueCore™ chip by CSR that is used in an EEG measurement device transmitter and a stimulation coil transmitter and is a Bluetooth stack structure used in an embodiment of the present invention. Reference numeral ‘902’ shows use of a microprocessor and bluetooth. In reference numerals ‘903’ and ‘904’ in an embodiment of the present invention, connection of an EEG measurement device and an initialization PC between a microcontroller and a Bluetooth module in a stimulation apparatus, and data transmission operation are performed using an HCI packet and an L2CAP packet. Reference numeral ‘904’ shows a stimulation apparatus connected with a microprocessor, reference numeral ‘905’ shows a PC or a cell phone, and a connection process of a microprocessor of reference numeral ‘906’ and Bluetooth of reference numeral ‘907’ is shown.

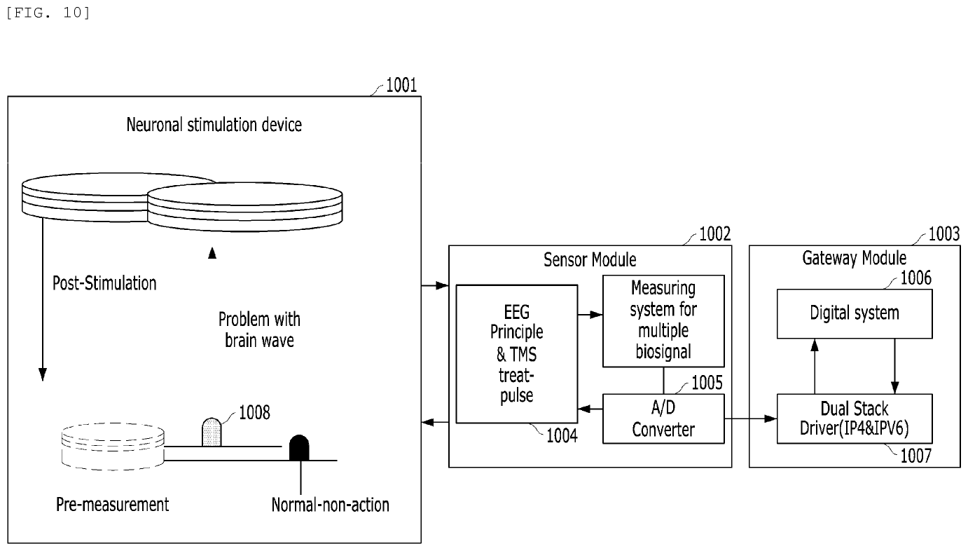

FIG. 10 is a diagram showing an example of a first operation of the cranial nerve stimulation apparatus of FIG. 1.

As shown in FIG. 10, a cranial nerve stimulation apparatus 1001 communicates with a module that processes a biological signal measured by a sensor module 1002 and a treatment stimulation coil, and may include a digital system 1006 converting analog signals measured by a (first) sensor module 1002 and a (second) sensor module (or gateway module) 1003 into digital signals by quantizing the analog signals. In reference numeral ‘1004’, in order to measure a cranial nerve stimulation apparatus of a treatment pulse suitable for a cerebral cortex variant of mammalian, a pulse signal EEG and stimulation apparatus is measured, and reference numeral ‘1005’ corresponds to a sensor module that can perform signal processing to them after A/D converting and then transmit them through a Bluetooth radio communication method, in which a frequency range and width, a gain bandwidth for a frequency width, etc. that are inherent characteristics are different, so signal processing is separately performed. Further, biological signals obtained by the sensor module are transmitted to a gateway using Bluetooth and the gateway module 1003 receives biological signals through Bluetooth communication and then transmits them using IPv4 or IPv6-based TCP/IP communication of reference numeral ‘1007’.

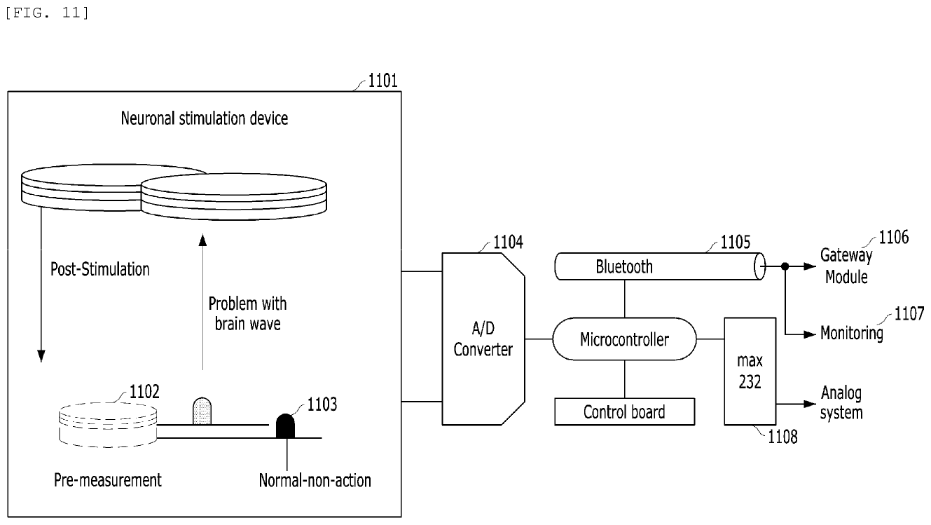

FIG. 11 is a diagram showing another example of the configuration of the cranial nerve stimulation apparatus of FIG. 1.

As shown in FIG. 11, reference numeral ‘1004’ is an A/D converter, a Micro Controller Unit (MCU) of reference numeral ‘1005’ may be configured using ATMEL and MSP430 (Texas Instrument, Co., Ltd, Texas, USA), in which the MCU is configured by a 48 KByte flash memory and a 12 bit ADC with 6 channels, and the ADC converts EEG and EMG signals, which are analog signals output from a sensor module, into digital signal using three of six channels in the MSP430, in which a sampling ratio per channel is 512 Hz and the resolution may be set as 12 bit. Reference numeral ‘1107’ shows monitoring and reference numeral ‘1108’ shows MAX 232 IC that can transmit a signal in an analog type.

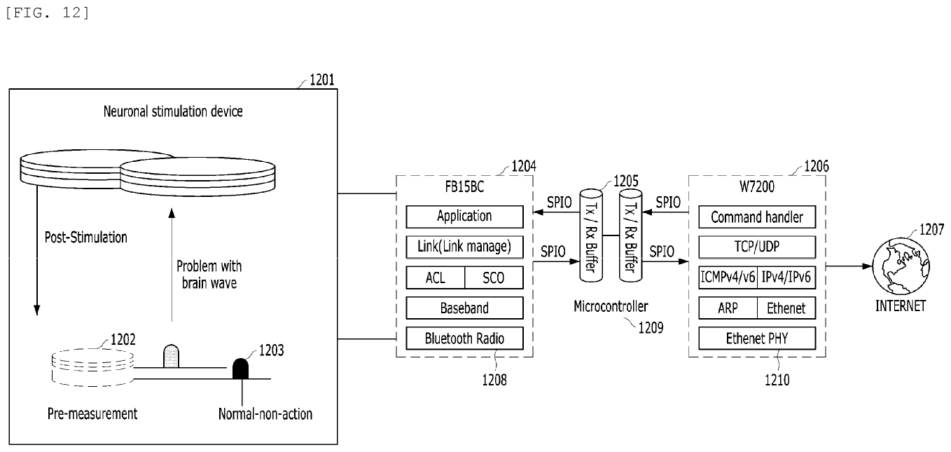

FIG. 12 is a diagram showing another example of the configuration of the cranial nerve stimulation apparatus of FIG. 1.

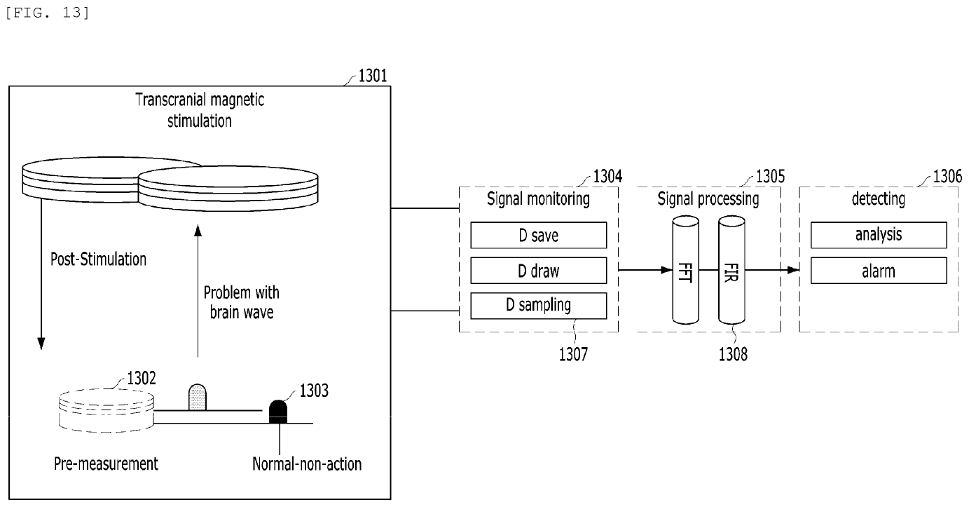

As shown in FIG. 12, in order to construct a high-reliability WBAN environment, a biological signal transmitted from Bluetooth of a sensor module 1201 may be designed to be able to communicate with a gateway on TCP/IP and may be designed to operate even in not only an IPv4, but also in an IPv6 environment. An IPv4/IPv6 used for the gateway is W7200 (Wiznet), thus the structure of the stack of the W7200 of reference numeral ‘1206’ is the same as reference numeral ‘1210, the left stack structures of reference numeral ‘1204’ and the reference numeral ‘1208’ are Bluetooth standard stack structures, and the right stack structures of reference numeral ‘1206’ and reference numeral ‘1210’ are IPv4/IPv6 Dual stack structures. FIG. 13 is a diagram showing two kinds of signal processing processes for signal analysis.

As shown in FIG. 13, in an embodiment of the present invention, reference numeral ‘1304’ shows signal monitoring, reference numeral ‘1305’ shows signal processing, and reference numeral ‘1306’ shows a detecting function, and two kinds of signal processing processes for signal analysis are shown in FIG. 13. In reference numeral ‘1308’, the first signal processing process is a Fast Fourier Transform (FFT) process, and more parameters and information that can be used for diagnosis are extracted by converting time-series data in a time domain in reference numeral ‘1308’ into a frequency domain. As the second signal processing processes, Finite Impulse Response (FIR) can be applied, in which FIR is a digital filter, which can be achieved as software, and can effectively compensate for the defects of an analog filter, so it can show high performance in comparison to a hardware filter.

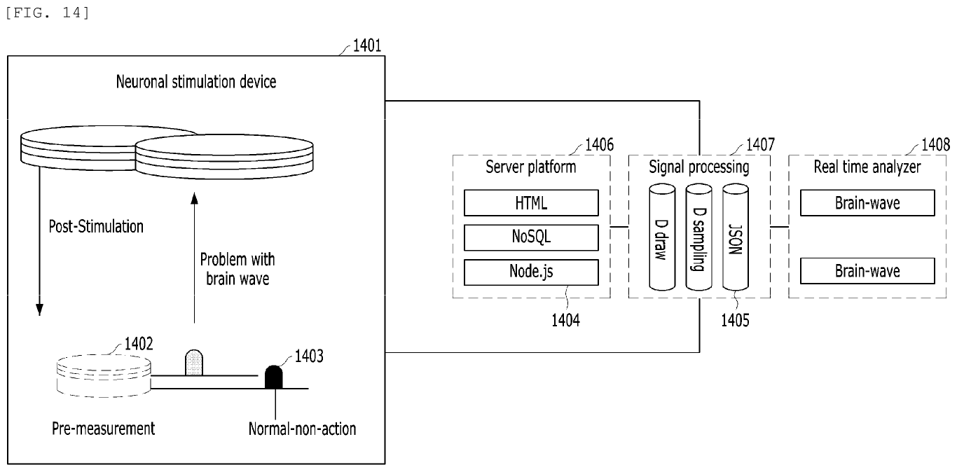

FIG. 14 is a diagram showing the structure of a web-based monitoring system.

As shown in FIG. 14, there are provided reference numerals ‘1401’ to ‘1403’ for each function of a sensor module and reference numeral ‘1404’ shows one that can be configured by HTML, NoSQL, Node.js of a server platform. Reference numeral ‘1405’ shows one configured by data draw, data sampling, JSON, etc. Reference numeral ‘1406’ shows a server platform, in which a server environment is suitable for large-size data analysis, a web server and a database server are constructed, and Node.js that is a distributed web server based on events is used as a web server. In reference numeral ‘1047’, JSON, data sampling, and datadraw are discriminated for file processing and the data processing method uses a JavaScript Object Notation (JSON) type, which is because JSON is a data type that can quickly process Extensible Markup Language (XML) and small data. Since it has less function than XML, data parsing is quick, and since the format is simple, so it is suitable for the mobile environment.

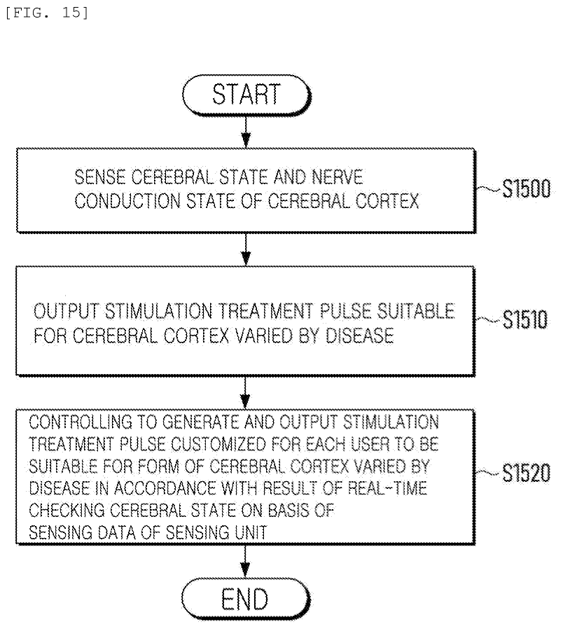

FIG. 15 is a flowchart showing a driving process of a cranial nerve stimulation apparatus according to an embodiment of the present invention.

For the convenience of description, referring to FIG. 15 with FIG. 1, the cranial nerve stimulation apparatus 90 according to an embodiment of the present invention may be primates dementia treatment apparatus can sense a cerebral state and a nerve conduction state of a cerebral cortex (S1500). In order to sense a cerebral state and a nerve conduction state, an EEG sensor and an EMG sensor may be used.

Further, the cranial nerve stimulation apparatus 90 outputs a stimulation treatment pulse suitable for the form of a cerebral cortex varied by a disease (S1510). Since the cerebral states and the progression state of a disease depend on patients, the stimulation treatment pulse is output in types corresponding to patient in consideration of the problem.

As described above, the cranial nerve stimulation apparatus 90 checks a cerebral state in real time, that is, without disconnection of data on the basis of sensing data, and generates and outputs a simulation treatment pulse corresponding to patients to fit to the form of a cerebral cortex varied by a disease in accordance with the checking result (S1520).

The cranial nerve stimulation apparatus 90 may use a DC superposition circuit to maximize treatment, and can provide optimized treatment for each dementia patient by adjusting the depth and area of a brain cortex in accordance with the brain size by applying the DC superposition network.

Other than the above description, the cranial nerve stimulation apparatus 90 can perform various operations, and other details were sufficiently described above, so the above description is referred to.

Even through all components of embodiments of the present invention are combined in one unit or operated in combination in the above description, the present invention is not limited thereto. That is, the all components may be selectively combined and operated within the scope of the present invention. Further, although all the components may be implemented as discrete hardware, some or all of the components may be selectively combined as computer programs having program modules that perform some or all of functions combined in one or several items of hardware. Codes and code segments of the computer programs may be easily inferred by those skilled in the art. The computer programs may be stored in a nontransitory computer readable media and read out and executed by a computer, thereby achieving embodiments of the present invention.

The nontransitory computer readable media is not a media that stores data for a short time such as a register, a cache, and a memory, but a media that can semipermanently store data and can be read out by a device. In detail, the programs may be stored and provided in a nontransitory computer readable media such as a CD, a DVD, a hard disk, a blueray disc, a USB, a memory card, and a ROM.

Although exemplary embodiments of the present disclosure were illustrated and described above, the present disclosure is not limited to the specific exemplary embodiments and may be modified in various ways by those skilled in the art without departing from the scope of the present disclosure described in claims, and the modified examples should not be construed independently from the spirit of the scope of the present disclosure.

What is claimed is:

1. A primates dementia treatment apparatus comprising:
a sensing unit including sensors that sense a cerebral state and a nerve conduction state of a cerebral cortex;
a stimulation pulse output unit outputting a stimulation treatment pulse suitable for a cerebral cortex varied by a disease; and
a controller controlling the stimulation pulse output unit to generate and output a stimulation treatment pulse customized for each user to be suitable for a form of a cerebral cortex varied by a disease in accordance with a result of real-time checking of the cerebral state on the basis of sensing data of the sensing unit.

2. The primates dementia treatment apparatus of claim 1, wherein the controller checks a form of a cerebral cortex and a disease progression state as the cerebral state and the nerve conduction state on the basis of the sensing data.

3. The primates dementia treatment apparatus of claim 1, wherein the sensing unit includes an electroencephalogram (EEG) sensor that senses the cerebral state and an electromyography (EMG) sensor that senses the nerve conduction state.

4. The primates dementia treatment apparatus of claim 1, wherein the stimulation pulse output unit includes a DC superposition network that superposes and delays the stimulation treatment pulse to fit to a depth and an area of a cerebral cortex of each user in order for treatment according to a reduction of a brain size.

5. The primates dementia treatment apparatus of claim 4, wherein the stimulation pulse output unit further includes a stimulation coil unit that discharges the stimulation treatment pulse of the DC superposition network under the control of the controller.

6. A method of driving a primates dementia treatment apparatus, the method comprising:
sensing a cerebral state and a nerve conduction state of a cerebral cortex by means of a sensing unit;
outputting a stimulation treatment pulse suitable for a cerebral cortex varied by a disease by means of a stimulation pulse output unit; and
controlling the stimulation pulse output unit to generate and output a stimulation treatment pulse customized for each user to be suitable for a form of a cerebral cortex varied by a disease in accordance with a result of real-time checking the cerebral state on the basis of sensing data of the sensing unit by means of a controller.

7. The method of claim 6, wherein the controlling includes checking a form of a cerebral cortex and a disease progression state as the cerebral state and the nerve conduction state on the basis of the sensing data.

8. The method of claim 6, wherein the sensing senses the cerebral state using an electroencephalogram (EEG) sensor and senses the nerve conduction state using an electromyography (EMG) sensor.

9. The method of claim 6, wherein the outputting of a stimulation treatment pulse includes superposing and delaying the stimulation treatment pulse to fit to a depth and an area of a cerebral cortex of each user in order for treatment according to a reduction of a brain size, using a DC superposition network of the stimulation pulse output unit.

10. The method of claim 9, wherein the outputting of a stimulation treatment pulse discharges the stimulation treatment pulse of the DC superposition network under the control of the controller, using a stimulation coil unit of the stimulation pulse output unit.
